(19) United States
(12) Patent Application Publication
(10) Pub. No.: US 2022/0008100 A1
(43) Pub. Date: Jan. 13, 2022

(54) CANNULAS WITH NON-CIRCULAR CROSS-SECTIONS, SYSTEMS, AND METHODS

(71) Applicant: INTUITIVE SURGICAL OPERATIONS, INC., Sunnyvale, CA (US)

(72) Inventors: Ronald G. LITKE, Sandy Hook, CT (US); Joseph P. ORBAN, III, Norwalk, CT (US); Justin KROM, Southington, CT (US); John W. ZABINSKI, Fremont, CA (US); Nathan VENSKYTIS, Woodbridge, CT (US)

(73) Assignee: INTUITIVE SURGICAL OPERATIONS, INC., Sunnyvale, CA (US)

(21) Appl. No.: 17/299,452

(22) PCT Filed: Dec. 4, 2019
(86) PCT No.: PCT/US2019/064416

§ 371 Date: Jun. 3, 2021

Related U.S. Application Data
(60) Provisional application No. 62/775,432, filed on Dec. 5, 2018.

(51) Int. Cl. A61B 17/34

(52) U.S. Cl. CPC A61B 17/3474; A61B 2017/3433; A61B 17/3421

(57) ABSTRACT

A cannula includes a tube having a central passage extending between a proximal end and a distal end of the tube along a longitudinal axis of the tube. A first cross section of the passage taken at or adjacent the distal end of the tube and in a plane normal to the longitudinal axis has a first cross-sectional shape, the first cross-sectional shape being non-circular. A second cross section of the passage taken through a portion of the tube proximal to the distal end of the tube and in a plane normal to the longitudinal axis has a second cross-sectional shape. The first cross-sectional shape is different from the second cross-sectional shape. Systems related to cannulas and tools for insertion through cannulas.

### CROSS-REFERENCE TO RELATED APPLICATIONS

This application claims priority to U.S. Provisional Application No. 62/775,432 (filed Dec. 5, 2018), the entire contents of which are incorporated by reference herein.

### TECHNICAL FIELD

Aspects of the present disclosure relate to cannulas for tools, such as surgical instruments. Cannulas according to the disclosure feature non-circular cross-sections and can accommodate flow of an insufflation gas between an inner wall of the cannula and an outer surface of a tool.

### INTRODUCTION

Cannulas may be used to guide, position, and/or support a tool, such as a surgical instrument, during a procedure, such as a surgical operation. For example, during some procedures, a cannula is positioned in a body wall, and a tool such as a surgical instrument is inserted through the cannula to access a subject site, such as a surgical site. Examples of such a procedure can include minimally invasive surgery carried out with a teleoperated tool. During such a procedure, the subject site may be insufflated with gas to facilitate the procedure by creating a space to carry out the procedure. Insufflation can also serve to prevent infection by preventing inflow or other intrusion of foreign material into the surgical site.

Insufflation pressure may be supplied through a space within the cannula between the cannula wall and the tool inserted through the cannula. The flow rate of gas through the cannula to the surgical site may limited by the cross-sectional area of a space between the outer surface of the tool inserted through the cannula and an inner surface of the cannula. Increasing the cross-sectional dimensions of the cannula relative to the tool can increase the cross-sectional area and thus increase the flow rate, but such an increase in the dimension of the cannula requires a corresponding increase in incision size to accommodate the larger cannula. Additionally, increasing cannula size relative to the tool size may provide less support for the end of the tool as the tool protrudes from the cannula and may, therefore, permit undesirable movement or vibration of the tool relative to the cannula.

A need exists for cannulas that facilitate a relatively large flow of insufflation gas without requiring a corresponding increase in incision size. A need also exists for such cannulas to provide adequate support for a tool end protruding from the cannula.

### SUMMARY

Embodiments of the present disclosure may solve one or more of the above-mentioned problems and/or may demonstrate one or more of the above-mentioned desirable features. Other features and/or advantages may become apparent from the description that follows.

In accordance with one aspect of the disclosure, a cannula includes a tube having a central passage extending between a proximal end and a distal end of the tube along a longitudinal axis of the tube. A first cross section of the passage taken at or adjacent the distal end of the tube and in a plane normal to the longitudinal axis has a first cross-sectional shape, the first cross-sectional shape being non-circular. A second cross section of the passage taken through a portion of the tube proximal to the distal end of the tube and in a plane normal to the longitudinal axis has a second cross-sectional shape. The first cross-sectional shape is different from the second cross-sectional shape.

In accordance with another aspect of the disclosure, a system includes a cannula with a tube having a longitudinal axis extending between a proximal end and a distal end of the tube and a passage extending between the proximal and distal ends of the tube. A distal end portion of the tube includes a portion of the passage with a first cross-sectional shape taken in a plane normal to the longitudinal axis and a proximal portion of the tube includes a portion of the passage with a second cross-sectional shape taken in a plane normal to the longitudinal axis. The system includes a tool comprising a shaft inserted within the passage. A circle inscribed within the first cross-sectional shape has a first diameter that defines a first clearance between an outer perimeter of the passage and the shaft of the tool when the tool is inserted in the passage. The second cross-sectional shape defines a second clearance, larger than the first clearance, between the outer perimeter of the passage and around the shaft of the tool when the tool is inserted in the passage. The first cross-sectional shape is different from the second cross-sectional shape.

In accordance with yet another aspect of the present disclosure, an apparatus includes a cannula tube with a proximal end, a distal end, and a first passage defined between the proximal and distal ends. An insufflation source fitting is at the proximal end of the tube. The insufflation source fitting comprises a second passage, and the passage of the insufflation source fitting joins the first passage of the tube. A cross section of tube is a polygon.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description serve to explain certain principles and operation. In the drawings,

FIG. 1 is a schematic side view of an embodiment of a system including a cannula according to the present disclosure.

FIG. 2 is a cross-sectional view of the cannula of FIG. 1.

FIG. 3 is another cross-sectional view of the cannula of FIG. 1.

FIG. 4 is a perspective view of another embodiment of a cannula according to the present disclosure.

FIG. 5 is a distal end view of the cannula of FIG. 4.

FIG. 6 is a perspective view of another embodiment of a cannula according to the present disclosure.

FIG. 7 is a distal end view of the cannula of FIG. 6.

FIG. 8 is a perspective view of another embodiment of cannula according to the present disclosure.

FIG. 9 is a distal end view of the cannula of FIG. 8.

FIG. 10 is a perspective view of another embodiment of a cannula according to the present disclosure.

FIG. 11 is a distal end view of the cannula of FIG. 10.

FIG. 12 is a distal end view of a cannula according to another embodiment of the present disclosure.

FIG. 13 is a distal end view of a cannula according to another embodiment of the present disclosure.

FIGS. 14A and 14B are distal end views of a cannula according to yet another embodiment of the present disclosure in partially- and fully-formed states.

FIG. 15 is a side view of another embodiment of a cannula according to an embodiment of the present disclosure.

FIG. 16 is a perspective view of a manipulating system according to an embodiment of the present disclosure.

FIG. 17 is a partial schematic view of an embodiment of a manipulator arm of a manipulating system according to the present disclosure with two electrosurgical instruments in an installed position.

FIG. 18 is a schematic view showing a cross-sectional shape of a distal end portion of a cannula tube according to another exemplary embodiment of the present disclosure.

FIG. 19 is a schematic view showing a cross-sectional shape of a distal end portion of a cannula tube according to yet another exemplary embodiment of the present disclosure.

### DETAILED DESCRIPTION

The present disclosure contemplates various embodiments of cannulas configured to facilitate flow of insufflation gas while maintaining positioning and support of a tool inserted through the cannula. Embodiments of the disclosure can allow for increased flow of insufflation gas to compensate for pressure loss due to, for example, leakage through sealing elements in the system, retrieval of samples from a subject site, evacuation of the subject site, or other conditions that allow loss of pressure.

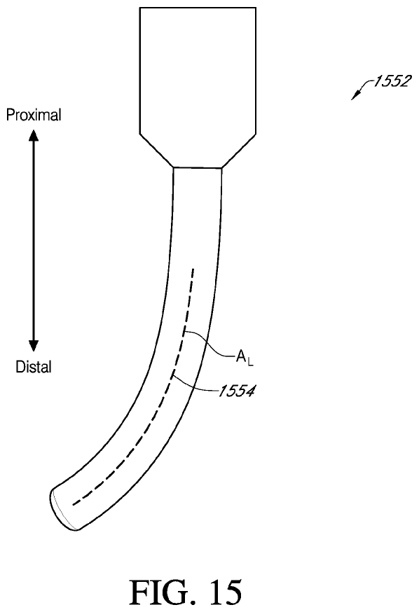

In various embodiments, a central passage of a cannula according to the present disclosure has a non-circular shaped cross section at or adjacent to a distal end portion of the cannula. Portions of the cannula with the non-circular passage cross section maintain contact with a tool inserted through the cannula's central passage while also providing clearance between the cannula's central passage and the tool and defining one or more flow paths around the tool within the cannula's central passage. Other portions of the cannula proximal to the distal end portion can comprise portions of the central passage having a round cross section, or a cross section shaped differently from a cross section of the passage at or adjacent to the passage at the distal end portion, and/or a larger shaped cross section than the cross section of the passage at or adjacent to the distal end portion of the cannula to provide greater clearance for inserting the tool within the central passage of the cannula. For example, greater clearance between the tool and central passage of the cannula in portions of the cannula proximal to the distal end portion can allow insertion of the tool without the tool binding within the cannula. Such clearance can be particularly useful for insertion of tools within curved cannulas, such as the cannula shown in FIG. 15.

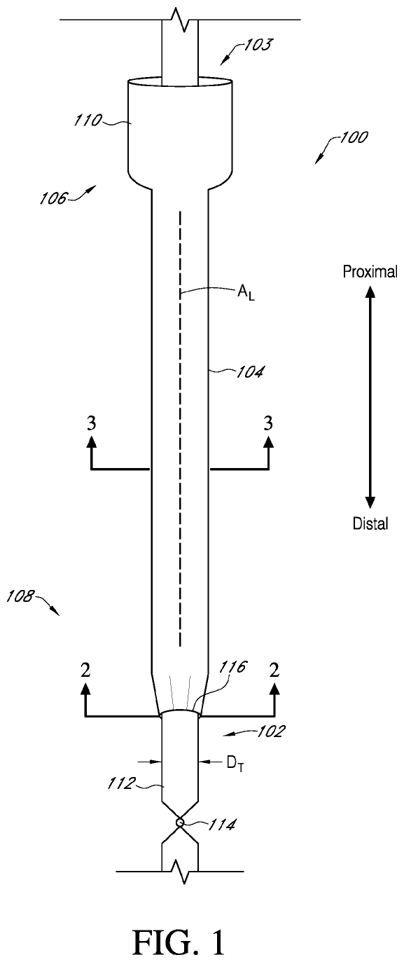

For example, in some embodiments of the present disclosure, the non-circular cross section of the central passage of the cannula's distal end portion defines at least one inscribed circle. That is, an inscribed circle diameter is defined by a circle passing through the radially innermost locations of an interior wall of the distal end portion of the cannula. The diameter of the inscribed circle is larger than an outer diameter of a tool inserted within the central passage of the cannula, and the difference between the diameter of the inscribed circle and the outside diameter of the tool is a clearance between the tool and the interior cannula wall that defines the central passage of the distal end portion of the cannula. A portion of the central passage of the cannula proximal to the distal end portion can have, as described above, a circular (or other shaped) cross section. This proximal portion of the central passage of the cannula has a dimension (such as of an inner diameter or inscribed circle) with a clearance between the tool and the interior wall defining the central passage of the cannula larger than the clearance at the distal end portion. The increased clearance in the central passage proximal to the distal end portion of the cannula enables the tool to be inserted through the cannula without binding, while the smaller clearance at the distal end portion of the cannula between the radially innermost locations of the non-circular cross section provides support for the tool to maintain the tool position. As used herein and shown in FIG. 1, the distalmost end of the cannula is the end of the cannula through which the tool or instrument exits the cannula to enter a subject site.

In various embodiments, the non-circular cross section of the central passage of the distal end portion of the cannula can include an elliptical cross section, an oval cross section, or a polygonal cross section. Polygonal cross sections, according to the present disclosure, can optionally comprise polygons with rounded vertices such as radiused vertices, polygons with pointed vertices, polygons with straight sides, polygons with curved sides such as Reuleaux polygons, or other configurations and all combinations thereof. Rounded configurations of these shapes can facilitate insertion of the cannula into a body wall and can facilitate manufacturing the cannula. Polygonal cross sections according to embodiments of the disclosure can have three or more sides, and these cross sections can include triangles, squares, pentagons, hexagons, heptagons, octagons, etc. Cross sections according to other embodiments can include one or more multiple concave, convex, and/or flat (not convex or concave) wall segments around a perimeter, such as a lobular cross-section. In various exemplary embodiments, the multiple wall segments may be alternating.

In various embodiments, an outer perimeter of the distal end portion having the non-circular-shaped passage cross section is equal in length to an outer perimeter of the proximal portion of the cannula with the circular-shaped passage cross section. Accordingly, the size of incision required for insertion of the cannula through a body wall to a subject site is determined by the diameter of the proximal portion of the cannula with the circular-shaped passage cross section, and the distal end portion with the non-circular-shaped passage cross section provides additional flow of insufflation gas without requiring a larger incision. Accordingly, compared to cannulas in which a distal end portion has a circular-shaped passage cross section, embodiments of the disclosure provide greater flow of insufflation gas, without requiring any corresponding increase in incision size.

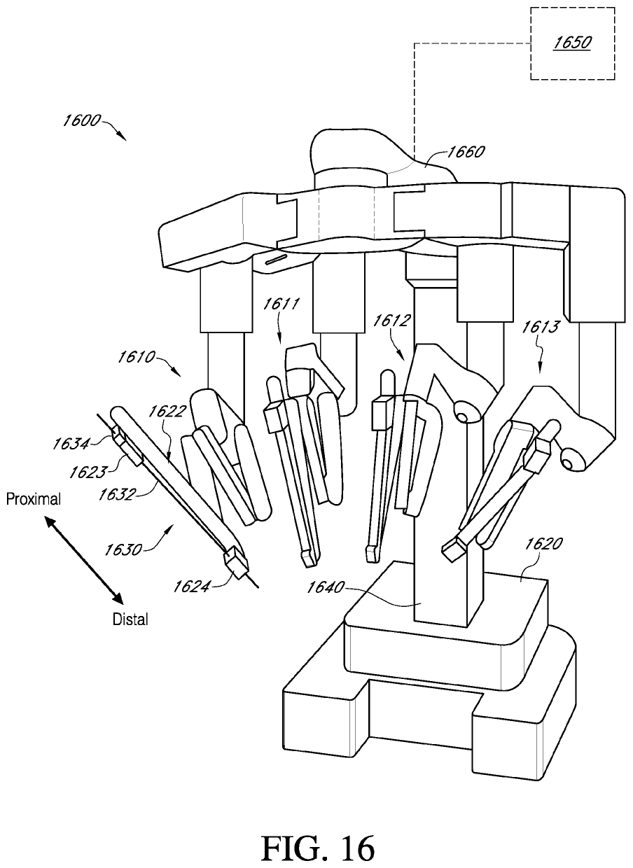

Referring now to FIG. 1, an embodiment of a cannula 100 and tool 102 is shown. The cannula 100 includes a cannula tube 104 having a proximal end portion 106 and a distal end portion 108. The proximal end portion 106 of the cannula tube 104 comprises a bowl 110, which can optionally be configured for coupling to a manipulating system, such as manipulating system 1600 shown in FIG. 16 and discussed below. The cannula tube 104 has a central passage 103 through which the tool 102 can be inserted. In the installed position of the tool 102 within the cannula 100 as shown in FIG. 1, a distal end 112 of the tool 102 protrudes beyond the cannula and includes one or more end effectors (not shown) such as, for example and not by way of limitation, forceps, shears, cautery tools such as a cautery hook, staplers, clip appliers, or other devices. The distal end 112 of the tool 102 can optionally further include one or more articulatable joints 114 to facilitate manipulation and positioning of the end effector of the tool 102.

A supply of pressurized insufflation gas is provided at the proximal end portion 106 of the cannula tube 104. Insufflation gas flows through the cannula tube 104 around the tool 102 and exits the distal end of the cannula tube 104 around the tool 102, as discussed further below. The insufflation gas may be provided by, for example, an insufflation gas source associated with a manipulating system, such as manipulating system 1600 discussed in connection with FIG. 16. Alternatively, any suitable insufflation gas source may be used, such as insufflation gas sources found in typical surgical theaters. Such an insufflation gas source can be connected to the cannula tube 104 by, e.g., a fitting, such as a Luer-type fitting, threaded connector, or other connector positioned near the proximal end portion 106 of the cannula tube 104.

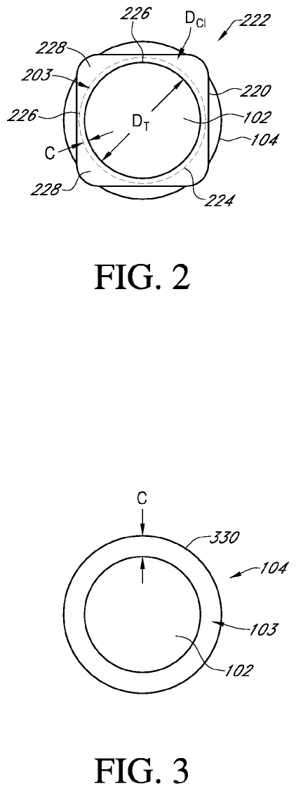

The central passage 103 at the distal end portion 108 of the cannula tube 104 comprises features configured to provide one or more flow paths around the tool 102 and out a distal opening 116 of the cannula when the tool 102 is inserted within the cannula 100, as shown in FIG. 1. As used herein, the term “distal end portion” can include a portion of the cannula tube including or adjacent to a distal end opening of the cannula tube. The distal end portion 108 also provides support to maintain the tool 102 in position within the distal end portion 108 of the cannula 100. For example, in embodiments of the present disclosure, the central passage 103 at the distal end portion 108 comprises a non-circular cross-sectional shape in a plane normal to the longitudinal axis A_{L} of the cannula tube 104 (i.e., the plane of FIG. 2). The non-circular cross-sectional shape of the central passage 103 defines one or more flow areas, in which an inner wall of the distal end portion 108 of the cannula tube 104 is spaced away from an outer diameter D_{T} of the tool 102, and one or more support areas, in which the inner wall of the distal end portion 108 of the cannula tube 104 is adjacent to or in contact with the outer diameter D_{T} of the tool 102. The flow areas form flow paths to direct insufflation gas supplied to the cannula tube to a subject site where the tool 102 is used.

For example, referring now to FIG. 2, the cross-sectional view of the distal end portion 108 of the cannula tube 104 indicated by section lines 2-2 in FIG. 1 is shown. The distal end portion 108 of the central passage 103 of the cannula tube 104 (FIG. 1) has a non-circular cross-sectional shape 220 in the view of FIG. 2. In the embodiment of FIG. 2, the non-circular cross-sectional shape 220 of central passage 203 is generally square with radiused vertices 222 (i.e., rounded corners). A circle 224 inscribed within the cross-sectional shape 220 has a diameter D_{CI}, and the tool 102 has an outer diameter D_{T}. A difference between the inscribed circle diameter D_{CI} and the outer diameter D_{T} of the tool 102 defines a clearance C₁ between the tool 102 and distal end portion 108 of the cannula 100. The clearance C₁ at the distal end portion can be chosen based on, for example, a desired degree of precision in position of the tool 102 relative to the cannula tube 104, among other possible factors. It is contemplated that the clearance at the distal end portion be in a range of, for example, from about 0.0005 (0.0127 mm) to about 0.031 inches (0.787 mm), while the clearance proximal of the distal end portion be in a range of, for example, from about 0.0285 inches (0.724 mm) to about 0.055 inches (1.40 mm). These dimensional ranges are provided by way of example only, and clearances of less than or greater than the above ranges are considered within the scope of the disclosure.

When the tool 102 is located within the cannula 100, as shown in FIGS. 1 and 2, the tool 102 contacts or nearly contacts only some parts of the distal end portion 108, identified in FIG. 2 to as portions 226 of the distal end portion 108 of the cannula tube 104 through which the inscribed circle 224 passes. In the embodiment of FIGS. 1 and 2, because the clearance exists between the tool 102 and the inscribed circle, the tool 102 does not contact all of the portions 226 simultaneously, and the tool 102 is free to move slightly within the inscribed circle 224 depending on the amount of clearance.

Referring still to FIG. 2, flow areas 228 are defined between the tool 102 and the non-circular cross-sectional shape of the central passage 203 at the distal end of the cannula tube 104. The flow areas 228 provide a flow path through which a fluid, such as insufflation gas, can flow into a subject site, such as, for example, an operation site within a patient's body.

In the embodiment of FIGS. 1 and 2, lateral and/or radial movement of the tool 102 is constrained by the size of the inscribed circle 224, while the flow areas 228 are defined by portions of the non-circular cross-sectional shape 220 that extend beyond the inscribed circle 224. Compared to conventional arrangements, such as those in which the distal end of the cannula central passage has a circular cross-sectional shape, embodiments of the present disclosure enable a close clearance between the tool 102 and the cannula tube 104 to maintain position of the tool 102 relative to the cannula, while also providing a relatively large flow area (such as flow areas 228) to facilitate a correspondingly large volume of gas flow. For example, some conventional tool and cannula designs feature a cannula with a circular distal end cross-section, and the flow area of such an arrangement is defined by a difference in diameter between the tool and the opening in the cannula. Any increase in cannula diameter at the distal end of the cannula to provide additional flow area results in a greater clearance between the tool and the cannula, and thereby permits greater movement between the tool and the cannula, compromising the accuracy and precision with which the tool can be maintained in position during a procedure. Embodiments of the present disclosure de-couple the relationship between flow area and clearance, facilitating accurate and precise positioning of the tool while also permitting relatively large flow rates of insufflation gas.

In the embodiment of FIG. 1, the non-circular cross-sectional shape 220 (FIG. 2) of the central passage 103 of the cannula tube 104 extends only along the distal end portion 108 of the cannula tube 104. The distance over which the non-circular cross-sectional shape 220 extends may be some portion of the overall length of the cannula tube 104. For example, in various embodiments, the non-circular cross-sectional shape 220 extends about one inch (25.4 mm) along the distal end portion 108 of the cannula tube 104. As a specific exemplary range, the non-circular cross-sectional shape extends a distance along the distal end portion 108 of the cannula tube in a range of from about 0.75 inches (19.1 mm) to about 1.25 inches (31.6 mm). In other embodiments, the non-circular cross-sectional shape can optionally extend less than 0.75 inches or more than 1.25 inches along the distal end portion 108 of the cannula tube 104.

The central passage 103 of the cannula tube 104 can optionally have a different cross-sectional shape along portions of the cannula tube 104 proximal of the distal end portion 108. In some embodiments, clearance between the cannula tube 104 and the tool 102 can optionally be larger proximal of the distal end portion 108 to prevent the tool 102 from binding within the cannula tube 104 as the tool 102 is inserted within the central passage 103 of the cannula tube 104, especially in embodiments that include curved cannula tubes (such as that discussed in connection with FIG. 15).

For example, referring now to FIG. 3, a cross-sectional view taken along line 3-3 in FIG. 1 is shown. The central passage 103 of the cannula tube 104 has a circular cross-sectional shape 330 that defines a clearance C₂ between the cannula tube 104 and the tool 102. In the embodiment of FIGS. 1-3, the clearance C₂ is greater than the clearance C₁ between the tool 102 and the inscribed circle 224 (FIG. 2) of the non-circular cross-sectional shape 220. This increased clearance can facilitate insertion and removal of the tool 102 within the cannula tube 104 without binding of the tool 102 within the cannula tube 104, without compromising the positioning of the tool 102 when the tool 102 is positioned within the cannula tube 104, because the distal end portion 108 of the cannula tube 104 provides accurate and precise locating of the tool 102. Because the circular cross-sectional shape 330 of the central passage 103 of the cannula tube 104 proximal of the distal end portion 108 has the relatively large clearance C₂ around the tool 102, the flow area through portions of the central passage 103 of the cannula tube 104 proximal of the distal end portion 108 is at least as large as the flow area defined by the flow areas 228 defined at the distal end portion 108 of the cannula tube 104. In this way, a relatively high and uniform flow rate is supported along the entire length of the cannula tube 104. For example, in testing conducted by the inventors, flow rates through the cannula of 50% greater to 77% greater than conventional designs were realized, depending on the diameter of the tool inserted within the cannula.

While the cross-sectional shape 330 shown in FIG. 3 associated with the embodiment of FIGS. 1-3 is circular, any other cross-sectional shape, such as polygons, ellipses, irregular shapes, or other cross-sectional shapes are within the scope of the disclosure. One factor to be considered in choosing the cross-sectional shape of the central passage 103 of the cannula tube 104 proximal of the distal end portion 108 is provision of sufficient clearance so the tool 102 to be inserted or withdrawn through the cannula tube 104 without binding in the cannula tube 104.

In various embodiments of the present disclosure, a perimeter dimension (e.g., length) of the outer surface of the cannula tube is consistent along the length of the cannula tube. For example, in the embodiment of FIGS. 1-3, a perimeter dimension of distal end portion 108 encompassing the non-circular cross-sectional shape 220 of the central passage of the cannula tube is approximately equal (e.g., similar or substantially identical) to a perimeter dimension of the cannula tube 104 encompassing the circular cross-sectional shape 330 of the central passage. Because the perimeter dimensions are similar or the same between the portion with the non-circular cross-sectional shape 220 of the central passage and the circular cross-sectional shape 330 of the central passage, the distal end portion 108 can be inserted into an incision in, e.g., a patient's body, without requiring the incision be enlarged beyond the size required based on the circular cross-sectional shape 330.

In various embodiments of the present disclosure, the non-circular cross-sectional shape 220 of the central passage is formed by a process such as stamping, die forming, or other processes. In one example embodiment, the cannula tube 104 is formed of tubular stock with the desired diameter and made of a relatively ductile metal alloy, such as, for example, 304 stainless steel. The cannula tube 104 is then cut to a desired length, and the non-circular cross-sectional shape 220 is stamped, die-formed, or otherwise imparted to the distal end portion 108 of the cannula tube 104. In other embodiments, the cannula can optionally be molded, such as from a moldable polymer material, or cast from metal or polymer materials.

In some embodiments of the disclosure, the forming process used to impart the non-circular cross-sectional shape 220 to the central passage at the distal end portion 108 of the cannula tube 104 does not appreciably change the perimeter dimension of the cannula tube 104, and therefore, the portion of the cannula tube 104 with the non-circular cross-sectional shape 220 of the central passage exhibits a perimeter dimension substantially equal to a perimeter dimension (e.g., circumference) of the cannula tube 104 portion having the circular cross-sectional shape 330 of the central passage. In other embodiments, one or more processes used to form the non-circular cross-sectional shape 220 of the central passage of the distal end portion of the cannula tube 104 can optionally have a stretching or shrinking effect on the material of the cannula tube 104, and consequently, result in a small difference between the perimeter of the cannula tube 104 portion having the non-circular cross-sectional shape 220 of the central passage and the portion of the cannula tube 104 having the circular cross-sectional shape 330 of the central passage.

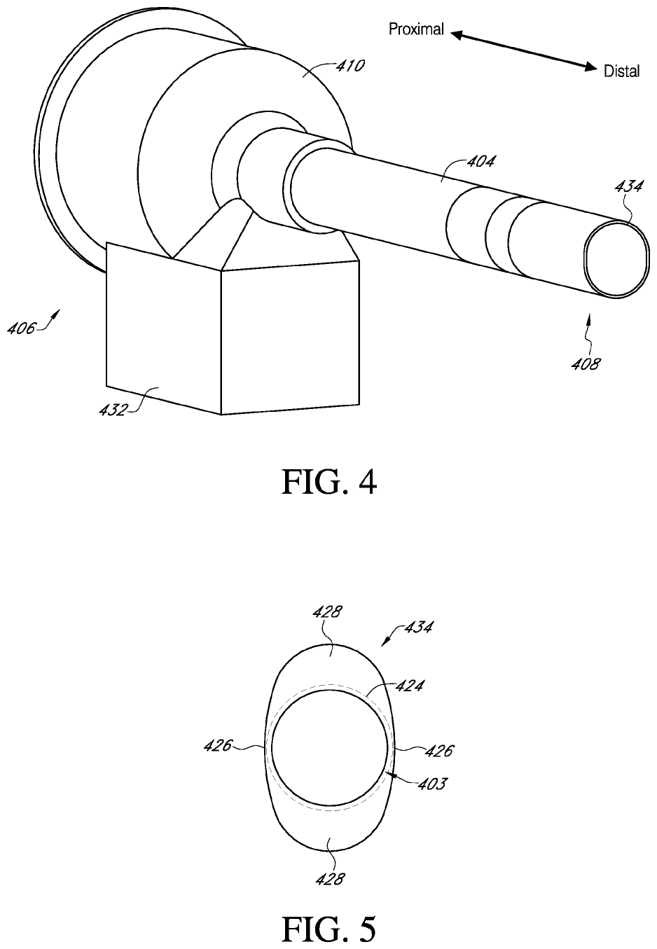

A variety of different cross-sectional shapes can be used at the distal end portion of the cannula tube. FIGS. 4-14 illustrate examples of other shapes that can be used according to various embodiments of the present disclosure.

Referring now to FIGS. 4 and 5, another embodiment of a cannula 400 according to the present disclosure is shown. Cannula 400 includes a cannula tube 404, a proximal portion 406 with a bowl portion 410, and a distal end portion 408. The proximal portion 406 includes an attachment portion 432 configured to couple with, e.g., a portion of a manipulating system such as manipulating system 1600 discussed in connection with FIG. 16. The distal end portion 408 has a central passage 403 with a non-circular cross-sectional shape 434 that is oval-shaped. Referring to FIG. 5, a circle 424 inscribed within the oval-shaped cross section 434 defines contact portions 426 at which the tool 102 can contact the cannula tube 404. Flow areas 428 define one or more passageways between the tool 102 and the cannula tube 404 through which insufflation gas flows into the surgical site.

As with the embodiment of FIGS. 1-3, the central passage 403 of the cannula tube 404 features a circular cross-sectional shape proximal to the distal end portion 408. The perimeter (i.e., circumference) of the cannula tube portion having the central passage with the circular cross-sectional shape proximal of the distal end portion 408 is substantially equal to the perimeter of the cannula tube portion having the central passage with the oval-shaped cross section 434.

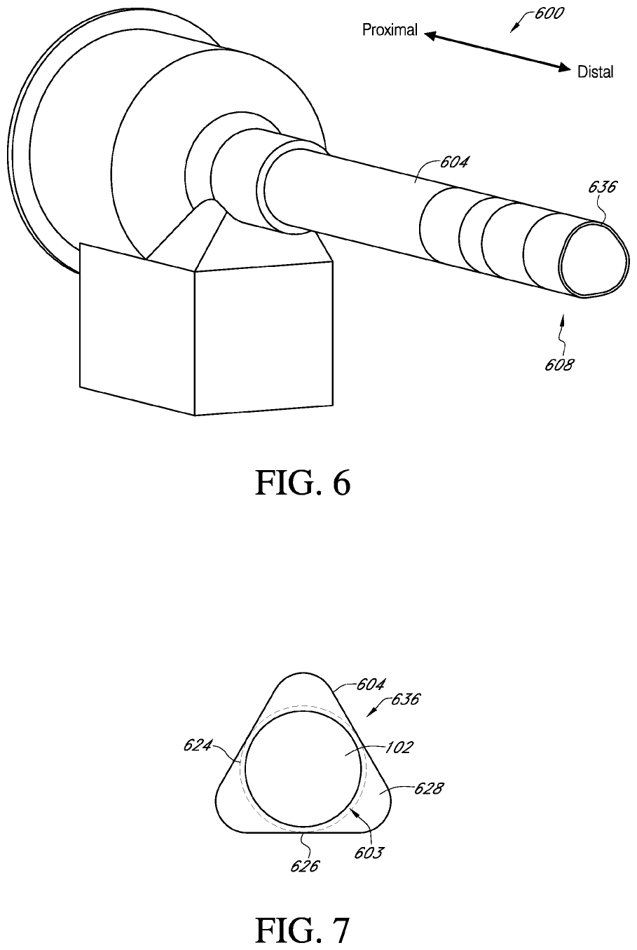

Referring now to FIGS. 6 and 7, another embodiment of a cannula according to the present disclosure is shown. In FIGS. 6 and 7, the cannula 600 is similar in many respects to the cannulas described in connection with FIGS. 1-5, but the cannula tube 604 includes a distal end portion 608 with a central passage 603 having a non-circular cross-sectional shape 636 that is generally triangular. A circle 624 inscribed within the non-circular cross-sectional shape 636 thus defines three contact portions 626 at which the tool 102 can contact the cannula tube 604 at the distal end portion 608. That is, the contact portions 626 are configured to contact the tool 102 when the tool 102 is inserted within the cannula tube 604. Three flow areas 628 define areas between the cannula tube 604 and the tool 102 through which an insufflation gas can flow into a surgical site. Like the embodiment of FIGS. 1-3, vertices of the triangular cross-sectional shape are radiused to provide a smooth outer surface of the cannula tube 604.

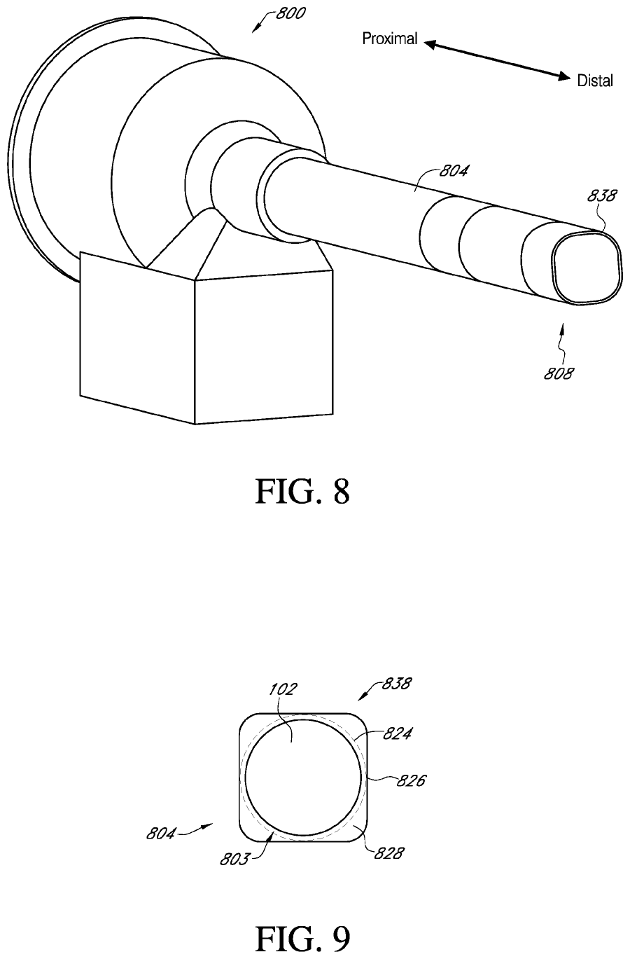

Referring now to FIGS. 8 and 9, an embodiment of a cannula 800 similar to the embodiment described in connection with FIGS. 1-3 is shown, in that both embodiments have a distal end portion with a four-sided cross-sectional shape. The cannula tube 804 includes a distal end portion 808 with a central passage 803 having a non-circular cross-sectional shape 838 that is generally square with radiused vertices. A circle 824 inscribed within the non-circular cross-sectional shape 838 defines four contact portions 826 at which the tool 102 can contact the cannula tube 804 at the distal end portion 808. Four flow areas 828 define areas between the cannula tube 804 and the tool 102 through which insufflation gas can flow into a surgical site. Other aspects of the embodiment of FIGS. 8 and 9 are likewise similar to that discussed in connection with FIGS. 1-3.

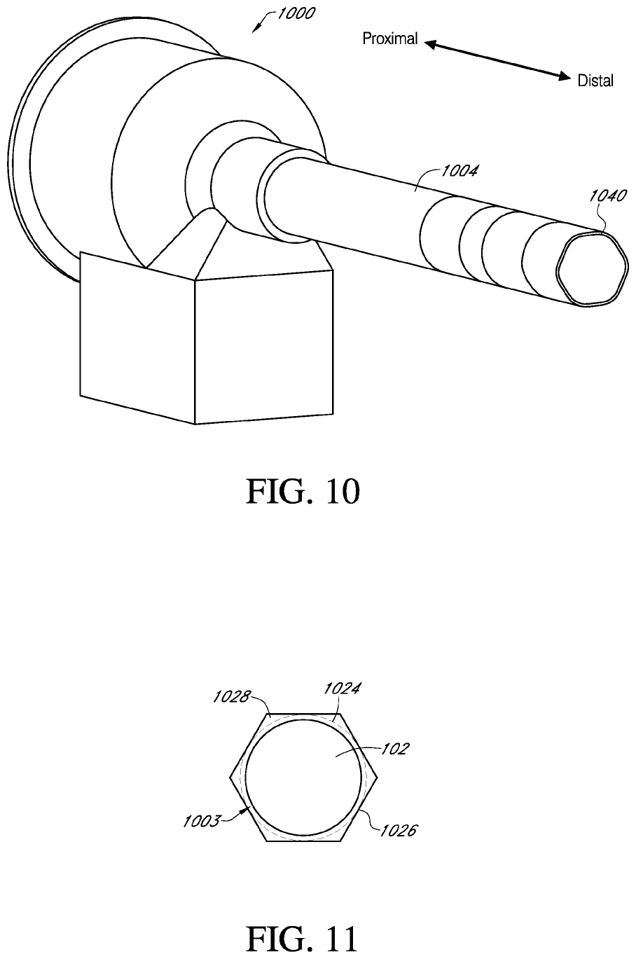

Referring to FIGS. 10 and 11, another embodiment of a cannula 1000 with a cannula tube 1004 is shown. Cannula tube 1004 includes a distal end portion 1008 with a central passage 1003 having a non-circular cross-sectional shape 1040 that is generally hexagonal. Accordingly, a circle 1024 inscribed within the non-circular cross-sectional shape 1040 defines six contact portions 1026 at which the tool 102 can make contact with the distal end portion 1008 of the cannula tube 1004, and six flow areas 1028 defining areas through which insufflation gas can flow through the cannula 1000 into a surgical site.

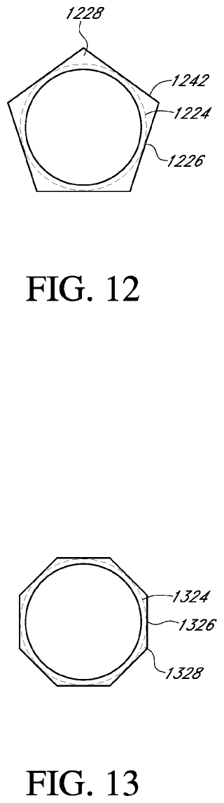

Non-circular cross-sectional shapes that can be used at the distal end of a cannula tube according to embodiments of the present disclosure are not limited to the shapes discussed in connection with FIGS. 1-11 above. For example, referring now to FIG. 12, yet another embodiment of a cross-sectional shape of a central passage of a distal end portion of a cannula tube is shown. In the embodiment of FIG. 12, the non-circular cross-sectional shape 1242 is generally pentagonal, and an inscribed circle 1224 defines five contact portions 1226 at which the tool 102 can make contact with the cannula tube, and five flow areas 1228 through which insufflation gas can flow through the cannula to a surgical site.

FIG. 13 shows an embodiment with a non-circular cross-sectional shape having eight sides (i.e., generally octagonal). In the embodiment of FIG. 13, an inscribed circle 1324 defines eight contact portions 1326 and eight flow areas 1328. It can be seen from FIG. 13 that, as the number of sides of the non-circular cross-sectional shape increases, the non-circular cross-sectional shape begins to approach a circular shape, and the area of the flow areas decreases. Without wishing to be bound to any particular theory or specific embodiments, the inventors have determined that a non-circular cross-sectional shape with 5 or 6 sides can provide a favorable compromise between the flow area available for insufflation gas and an overall cannula shape that interfaces acceptably with other system components, such as anchor devices that are coupled with the cannula by sliding over the distal end of the cannula.

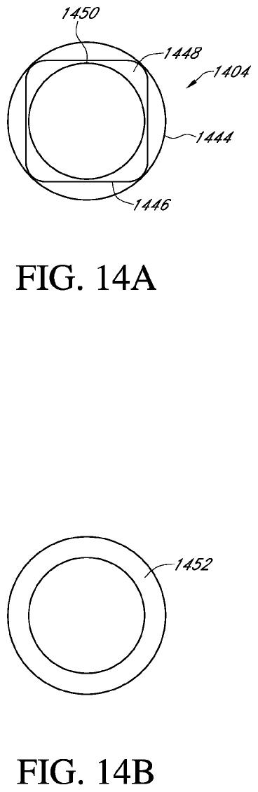

In some embodiments, a non-circular cross section of a central passage at a distal end of the cannula tube can optionally be formed by a material removal process, rather than a material deformation process. For example, referring now to FIGS. 14A and 14B, a cross-sectional view of a distal end portion of a cannula tube 1404 is shown. In FIG. 14A, the cannula tube 1404 has a circular outer surface 1444 and a non-circular inner surface 1446. In some embodiments, the non-circular inner surface 1446 can be formed from material stock having a circular inner surface by thinning the interior surface in chosen areas. For example, the cannula tube 1404 shown in FIG. 14A can be manufactured by starting with a tube 1452 having the cross section shown in FIG. 14B, i.e., circular inner and outer cross-sections. To arrive at the configuration shown in FIG. 14A, some portions of the thickness of the cannula tube 1404 are reduced to form flow areas 1448, while other portions of the thickness of the cannula tube 1404 are left close to or equal to the original thickness of the material stock of the tube 1452 to form contact portions 1450, which support the tool 102 in the manner discussed in greater detail in connection with the embodiment of FIGS. 1-3.

Referring again to FIGS. 4-14, it can be seen that the non-circular cross section at the distal end portion of the cannula results in two or more contact lines between the outer surface of the tool extending through the distal end portion and the inner surface of the non-circular cannula side wall. For the oval cross section shown in FIGS. 4 and 5, there are two contact lines, for the three-sided cross section shown in FIGS. 6 and 7 there are three contact lines, for the four-sided cross section shown in FIGS. 8 and 9 there are four contact lines, etc. With only two contact lines in the oval cross section, the tool is stabilized in only one cross-sectional dimension inside the distal end portion of the cannula. But with three or more contact lines surrounding the tool, the tool is stabilized in both cross-sectional dimensions inside the distal end portion of the cannula. In addition, since the outer surface of the tool contacts the inner surface of the cannula only along one or more contact lines, friction between the tool and the cannula side wall is minimized as the tool slides within the cannula.

In addition, it can be seen that for polygonal cross sections, the cannula side wall inner surface that contacts the tool's outer round surface optionally may be straight or curved, as long as the curve is sufficiently shallow to establish a contact line and not a contact area. As a geometric example, a circle that represents a tool's cross section can be inscribed within both an equilateral triangle and a Reuleaux triangle, with contact lines occurring where the circle touches each of the triangles' sides. If the shape of a side is altered to conform at least in part to the shape of the tool, however, then the contact area between the side wall and the tool is larger than a line, and friction between the tool and the side may be larger than if the contact is along a line. Persons of skill in the art will, of course, recognize the practical difference between this strictly geometric example and real-world objects, but the line versus area contact description nevertheless illustrates a principle of a cannula distal end portion that fully supports a tool extending through the distal end portion, that minimizes contact and friction between the tool and the cannula distal end portion, and that provides a sufficiently large fluid flow cross-sectional area between the cannula distal end portion and the tool.

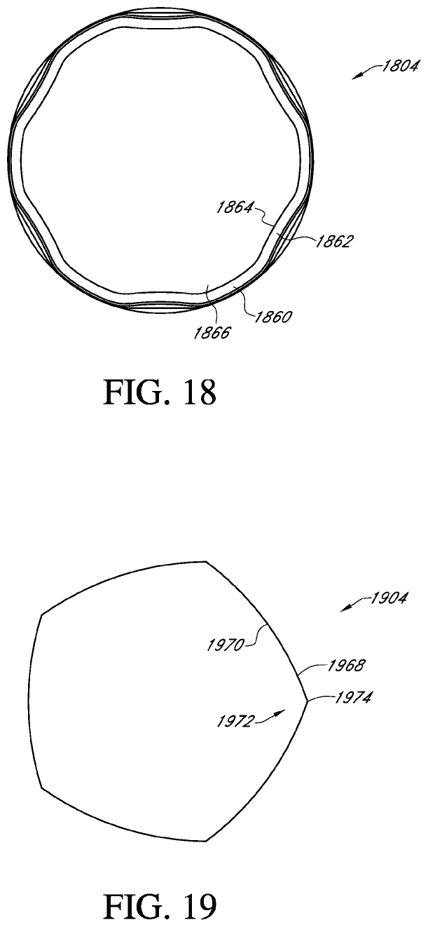

Other cross-sectional shapes of a cannula tube also are contemplated by the present disclosure. For example, the cannula tube can have a cross-sectional shape including alternating convex and concave wall segments around the circumference of the tube. Such a cross-sectional shape can be referred to as a lobular shape. FIG. 18 shows one exemplary embodiment of a cannula tube 1804 having a lobular cross-sectional shape having convex wall segments 1860 alternating with concave wall segments 1862, with the concavity/convexity being relative to an interior of the cannula tube 1804. The concave segments 1862 extend radially inward and thus define contact areas 1864 against which an instrument shaft (not shown) is supported, while the convex segments 1860 define flow regions 1866 around the instrument shaft to facilitate the flow of insufflation gas or other fluids, as discussed above. While the cross-sectional shape shown in FIG. 18 includes six convex portions 1860 alternating between six concave portions 1862, other numbers of convex and concave portions are within the scope of the disclosure. Further, other patterns, such as lobular patterns that do not alternate regularly, or patterns that include generally flat wall sections (not convex or concave) sections located between convex and/or concave portions, could be used.

FIG. 19 shows an embodiment of a cannula tube 1904 having a cross-sectional shape of a Reuleaux polygon. As used herein, a Reuleaux polygon is a polygon comprised of an arc segments. In this embodiment, the cannula tube 1904 has a cross-sectional shape of Reuleaux pentagon (i.e., a polygon having 5 sides, each being an arc segment). Each curved side (arc segment) 1968 defines a contact area 1970 at or near the midpoint of the curved side 1968 which provides support for an instrument shaft (not shown) inserted within the cannula tube 1904. Flow regions 1972 are defined between an instrument shaft inserted in the cannula tube 1904 and adjacent vertices 1974 and inner wall portions of the cannula tube 1904 proximate those vertices. While the example shown in FIG. 19 is a Reuleaux pentagon, other Reuleaux cross-sectional shapes having greater than five, or fewer than five, sides are within the scope of the present disclosure.

Referring now to FIG. 15, an embodiment of a cannula 1500 having a cannula tube 1504 with a curved section 1554 extending along its longitudinal axis A_{L} is shown. The various embodiments of cannula tubes and associated cross-sectional shapes described in connection with FIGS. 1-14 can be used in connection with straight cannulas, such as the cannula 100 in FIG. 1, or cannulas including one or more curved portions, such as the cannula 1500 in FIG. 15. Additionally or alternatively, in other embodiments of the present disclosure, a cannula can optionally include multiple (i.e., compound) curves. Embodiments of the present disclosure facilitate insertion and removal of tools (such as tool 102) within such cannulas without binding, enabling a relatively large flow of insufflation gas through the cannula distal end portion, and maintaining position of the tool 102 at the distal end portion of the cannula.

Embodiments described herein may be used, for example, with remotely operated, computer-assisted systems (such, for example, teleoperated surgical systems) such as those described in, for example, U.S. Pat. No. 9,358,074 (filed May 31, 2018) to Schena et al., entitled “Multi-Port Surgical Robotic System Architecture”, U.S. Pat. No. 9,295,524 (filed May 31, 2013) to Schena et al., entitled “Redundant Axis and Degree of Freedom for Hardware-Constrained Remote Center Robotic Manipulator”, and U.S. Pat. No. 8,852,208 (filed Aug. 12, 2010) to Gomez et al., entitled “Surgical System Instrument Mounting”, each of which is hereby incorporated by reference in its entirety. Further, the embodiments described herein may be used, for example, with a da Vinci® Surgical System, such as the da Vinci Si® Surgical System (model no. IS3000) or the da Vinci Xi® Surgical system, both with or without Single-Site® single orifice surgery technology, all commercialized by Intuitive Surgical, Inc. of Sunnyvale, Calif. Although various embodiments described herein are discussed with regard to surgical instruments used with a manipulating system of a teleoperated surgical system, the present disclosure is not limited to use with surgical instruments for a teleoperated surgical system. For example, various embodiments described herein can optionally be used in conjunction with hand-held, manual surgical instruments, or other surgical and non-surgical tools.

As discussed above, in accordance with various embodiments, surgical instruments of the present disclosure are configured for use in teleoperated, computer-assisted surgical systems (sometimes referred to as robotic surgical systems). Referring now to FIG. 16, an embodiment of a manipulating system 1600 of a teleoperated, computer-assisted surgical system, to which surgical instruments are configured to be mounted for use, is shown. Such a surgical system may further include a user control system, such as a surgeon console (not shown) for receiving input from a user to control instruments of manipulating system 1600, as well as an auxiliary system, such as a control/vision cart (not shown), as described in, for example, U.S. Pat. Nos. 9,358,074 and 9,295,524, incorporated above.

As shown in the embodiment of FIG. 16, a manipulating system 1600 includes a base 1620, a main column 1640, and a main boom 1660 connected to main column 1640. Manipulating system 1600 also includes a plurality of arms 1610, 1611, 1612, 1613, which are each connected to main boom 1660. Arms 1610, 1611, 1612, 1613 each include an instrument mount portion 1622 to which an instrument 1630 may be mounted, which is illustrated as being attached to arm 1610. Portions of arms 1610, 1611, 1612, 1613 may be manipulated during a surgical procedure according to commands provided by a user at the surgeon console. In an embodiment, signal(s) or input(s) transmitted from a surgeon console are transmitted to the control/vision cart, which may interpret the input(s) and generate command(s) or output(s) to be transmitted to the manipulating system 1600 to cause manipulation of an instrument 1630 (only one such instrument being mounted in FIG. 16) and/or portions of arm 1610 to which the instrument 1630 is coupled at the manipulating system 1600.

Instrument mount portion 1622 comprises a drive assembly 1623 and a cannula mount 1624, with a force transmission mechanism 1634 of the instrument 1630 connecting with the drive assembly 1623, according to an embodiment. Cannula mount 1624 is configured to hold a cannula 1636 through which a shaft 1632 of instrument 1630 may extend to a surgery site during a surgical procedure. The drive assembly 1623 contains a variety of drive and other mechanisms that are controlled to respond to input commands at the surgeon console and transmit forces to the force transmission mechanism 1634 to actuate the instrument 1630, as those skilled in the art are familiar with.

Although the embodiment of FIG. 16 shows an instrument 1630 attached to only arm 1610 for ease of viewing, an instrument may be attached to any and each of arms 1610, 1611, 1612, 1613. An instrument 1630 may be a surgical instrument with an end effector as discussed herein. A surgical instrument with an end effector may be attached to and used with any of arms 1610, 1611, 1612, 1613. The manipulating system 1600 can be operatively coupled to an insufflation gas source 1650, shown schematically in FIG. 16. The insufflation gas source 1650 can be or include, for example, a pressurized cylinder, pump, or other source. The embodiments described herein are not limited to the embodiment of FIG. 16 and various other teleoperated, computer-assisted surgical system configurations may be used with the embodiments described herein.

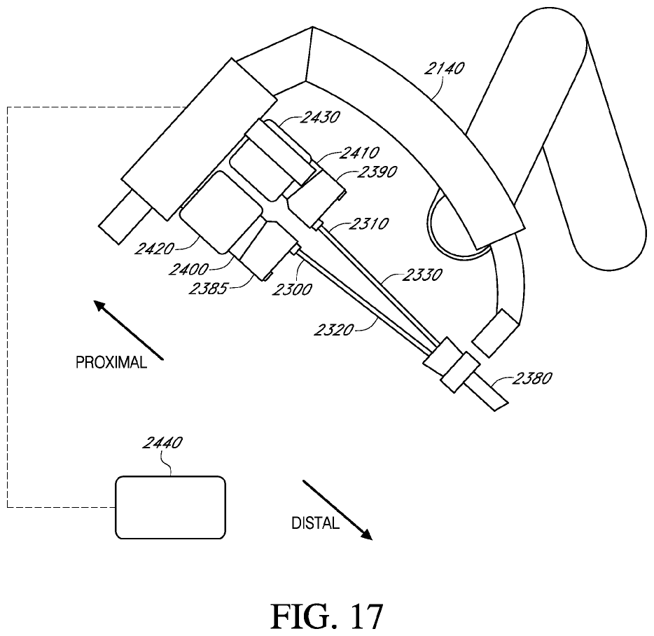

Other configurations of surgical systems, such as surgical systems configured for single-port surgery, are also contemplated. For example, with reference now to FIG. 17, a portion of an embodiment of a manipulator arm 2140 of a manipulating system with two instruments 2300, 2310 in an installed position is shown. A teleoperated robotic surgical system, including a manipulating system comprising manipulator arm 2140, may be configured according to the embodiments described in U.S. Patent App. Pub. No. US 2014/0128886 A1 (filed Nov. 1, 2013), to Holop et al. and titled FLUX DISAMBIGUATION FOR TELEOPERATED SURGICAL SYSTEMS, the disclosure of which is incorporated by reference herein. The schematic illustration of FIG. 17 depicts only two instruments for simplicity, but more than two instruments may be received in an installed position at a manipulating system as those having ordinary skill in the art are familiar with. Each instrument 2300, 2310 includes an instrument shaft 2320, 2330 that at a distal end has a moveable end effector or an endoscope, camera, or other sensing device, and may or may not include a wrist mechanism (not shown) to control the movement of the distal end.

In the embodiment of FIG. 17, the distal end portions of the instruments 2300, 2310 are received through a single port structure 2380 to be introduced into the patient. As shown, the port structure includes a cannula and an instrument entry guide inserted into the cannula. Individual instruments are inserted into the entry guide to reach a surgical site. Other configurations of manipulating systems that can be used in conjunction with the present disclosure can use several individual manipulator arms. In addition, individual manipulator arms may include a single instrument or a plurality of instruments. Further, an instrument may be a surgical instrument with an end effector or may be a camera instrument or other sensing instrument utilized during a surgical procedure to provide information, (e.g., visualization, electrophysiological activity, pressure, fluid flow, and/or other sensed data) of a remote surgical site.

Force transmission mechanisms 2385, 2390 are disposed at a proximal end of each shaft 2320, 2330 and connect through a sterile adaptor 2400, 2410 with drive assemblies 2420, 2430. Drive assemblies 2420, 2430 contain a variety of internal mechanisms (not shown) that are controlled by a controller (e.g., at a control cart of a surgical system) to respond to input commands at a surgeon side console of a surgical system to transmit forces to the force transmission mechanisms 2385, 2390 to actuate instruments 2300, 2310. The diameter or diameters of an instrument shaft, wrist mechanism, and end effector are generally selected according to the size of the cannula with which the instrument will be used and depending on the surgical procedures being performed. In various embodiments, a shaft and/or wrist mechanism has a diameter of about 4 mm, 5 mm, or 8 mm in diameter, for example, to match the sizes of some existing cannula systems. According to an embodiment, one or more of instruments 2300, 2310 may be inserted through a cannula as described above in communication with an insufflation gas source 2440, such as, for example, a pressurized cylinder, a pump, or other source of pressurized gas.

This description and the accompanying drawings that illustrate embodiments should not be taken as limiting. Various mechanical, compositional, structural, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the figures and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1-31. (canceled)

32. A cannula, comprising:
a tube comprising a proximal end, a distal end, a longitudinal axis extending between the proximal and distal ends of the tube, and a passage extending between the proximal and distal ends of the tube along the longitudinal axis of the tube;
wherein a first cross section of the passage taken at or adjacent the distal end of the tube and in a plane normal to the longitudinal axis has a first cross-sectional shape;
wherein the first cross-sectional shape comprises one of a polygonal shape and a lobular shape;
wherein a second cross section of the passage taken through a portion of the tube proximal to the distal end of the tube and in a plane normal to the longitudinal axis has a second cross-sectional shape; and
the second cross-sectional shape is different from the first cross-sectional shape.

33. The cannula of claim 32, wherein:
the first cross-sectional shape has a perimeter;
the second cross-sectional shape has a perimeter; and
the perimeters of the first and second cross-sectional shapes are of equal length.

34. The cannula of claim 32, wherein:
the first cross-sectional shape comprises a polygon and is selected from a triangle, a square, a pentagon, a hexagon, a heptagon, and an octagon.

35. The cannula of claim 34, wherein:
a circle inscribed within the first cross-sectional shape has a first diameter;
the second cross-sectional shape is a circle having a second diameter; and
the first diameter is smaller than the second diameter.

36. The cannula of claim 35, wherein:
portions of the passage outside the circle inscribed within the first cross-sectional shape are flow areas within the first cross-sectional shape; and
portions of the passage adjacent the circle inscribed within the first cross-sectional shape are contact portions of the first cross-sectional shape arranged to contact a tool inserted within the cannula.

37. The cannula of claim 32, wherein:
the tube comprises a portion curved along the longitudinal axis.

38. The cannula of claim 32, wherein:
the second cross-sectional shape is a circle.

39. The cannula of claim 32, wherein:
the first cross-sectional shape is a Reuleaux polygon.

40. The cannula of claim 32, wherein:
the first cross-sectional shape comprises a lobular shape having alternating convex and concave segments.

41. A system, comprising:
a cannula and a surgical tool inserted through the cannula;
wherein the cannula comprises a tube;
wherein the tube comprises a proximal end and a proximal end portion adjacent the proximal end, a distal end and a distal end portion adjacent the distal end, a longitudinal axis extending between the proximal and distal ends of the tube, and a passage extending between the proximal and distal ends of the tube;
wherein the distal end portion of the tube includes a portion of the passage with a first cross-sectional shape taken in a plane normal to the longitudinal axis;
wherein the proximal end portion of the tube includes a portion of the passage with a second cross-sectional shape taken in a plane normal to the longitudinal axis;
wherein the second cross-sectional shape is different than the first cross-sectional shape;
wherein the surgical tool comprises a shaft, and the shaft is inserted within the passage;
wherein the passage comprises an outer perimeter, and a circle inscribed within the first cross-sectional shape has a first diameter that defines a first clearance between the outer perimeter of the passage and the shaft of the surgical tool;
wherein the second cross-sectional shape defines a second clearance between the outer perimeter of the passage and the shaft of the surgical tool; and
wherein the second clearance is larger than the first clearance.

42. The system of claim 41, wherein:
the first cross-sectional shape comprises a polygon.

43. The system of claim 42, wherein:
the polygon comprises rounded vertices.

44. The system of claim 42, wherein:
the polygon is chosen from one of a triangle, square, pentagon, hexagon, heptagon, and octagon.

45. The system of claim 41, wherein:
the first cross-sectional shape comprises an oval.

46. The system of claim 41, wherein:
the second cross-sectional shape is circular.

47. The system of claim 41, wherein:
the first cross-sectional shape is non-circular.

48. The system of claim 41, wherein:
the system further comprises a plurality of flow areas defined between the shaft and the outer perimeter of the passage; and
each individual one of the plurality of flow areas is a corresponding individual flow path through the passage.

49. The system of claim 41, wherein:
the system further comprises an insufflation gas source; and
the insufflation gas source configured to supply pressurized insufflation gas to the passage of the tube.

50. The system of claim 41, wherein:
the first cross-sectional shape has a perimeter;
the second cross-sectional shape has a perimeter; and
the perimeters of the first and second cross-sectional shapes are of equal length.

51. An apparatus comprising:
a cannula tube comprising a proximal end, a distal end, an inner wall between the proximal and distal ends, and a first passage defined by the inner wall; and
an insufflation source fitting at the proximal end of the tube and comprising a second passage joining the first passage of the tube;
wherein a cross section of the tube transitions from a circular cross section proximate the proximal end of the tube to a polygonal cross section proximate the distal end of the tube.

52. The apparatus of claim 51, wherein:
the apparatus further comprises an instrument shaft extending through the first passage;
the instrument shaft comprises an outer surface; and
the outer surface of the instrument shaft contacts the inner surface of the tube along a contact line at the polygonal cross section of the tube.

53. The apparatus of claim 52, wherein:
the outer surface of the instrument shaft contacts the inner surface of the tube along a second contact line at the polygonal cross section of the tube.

54. The apparatus of claim 52, wherein:
the surface of the inner wall is straight at the contact line.

55. The apparatus of claim 52, wherein:
the surface of the inner wall is curved at the contact line.

56. The apparatus of claim 52, wherein:
the inner surface of the tube at the polygonal cross section comprises a pair of adjacent sidewalls joining at a vertex;
a fluid passage is defined between the outer surface of the instrument shaft and the pair of adjacent sidewalls; and
the second passage of the insufflation source fitting is joined to the fluid passage.
